(19) United States
(12) Patent Application Publication
(10) Pub. No.: US 2020/0251021 A1
(43) Pub. Date: Aug. 6, 2020

(54) "Protected Date Tag"

(71) Applicants: Mark Woodburn, Wexford, PA (US); Jim Gimbel, Wexford, PA (US)

(72) Inventors: Mark Woodburn, Wexford, PA (US); Jim Gimbel, Wexford, PA (US)

(21) Appl. No.: 16/782,367

(22) Filed: Feb. 5, 2020

Related U.S. Application Data
(60) Provisional application No. 62/801,157, filed on Feb. 5, 2019.

(51) Int. Cl. G09F 3/00; A61M 39/08; G09F 3/10; B32B 7/12; B32B 27/10; B32B 29/00

(52) U.S. Cl. CPC G09F 3/0295; A61M 39/08; G09F 3/10; B32B 7/12; B32B 27/10; B32B 2519/00; A61M 2205/6018; A61M 2205/0205; A61M 2205/583; B32B 2307/412; B32B 29/005

(57) ABSTRACT

A tag for medical tubing includes a transparent adhesive layer, a paper layer positioned on an upper surface of the transparent adhesive layer, and a non-transparent layer positioned on the upper surface of the transparent adhesive layer and an upper surface of the paper layer, in which a width of the paper layer is less than a width of the transparent adhesive layer such that a portion of the transparent adhesive layer is foldable to overlap the paper layer.

### CROSS REFERENCE TO RELATED APPLICATION

This application claims the benefit of U.S. Provisional Patent Application No. 62/801,157, filed Feb. 5, 2019, the disclosure of which is incorporated in its entirety by reference.

### BACKGROUND OF THE INVENTION

### Field of the Invention

The present disclosure is generally directed to tags for medical containers, tubing, or devices and, more particularly, to protected date tags for medical tubing.

### Description of Related Art

Currently, in the medical industry, a variety of different medical containers are used to hold and store powdered or liquid medicament. For example, syringes, test tubes, and the like are used to administer and/or store medicament. Further, catheter tubing and intravenous (IV) tubing is widely used to perform medical procedures and administer medicament to patients, respectively. A wide variety of adhesive labels are commercially available to attach to the catheter tubing and IV tubing to permit a physician to provide information regarding the specific medicament being administered to the patient, the date and time the medicament was administered to the patient, and/or the date and time when the IV tubing needs to be replaced or removed, among other information. Most of these adhesive labels are general purpose labels having an adhesive layer on one side and front surfaces adapted for written or typed identifications. It is also common to provide a date on or near the tubing inserted into the patient to indicate to a nurse or physician when the tube was inserted into the patient. This assists the nurse and physician in determining when the tubing should be removed or replaced. Typically, the date is written on a housing or gauge of the medical device that is used to supply medical fluid to the tubing. The adhesive label used on the housing or gauge is periodically removed, thereby losing the date information due to deterioration of the writing on the label. The original date that was written on the label is often lost, leaving the nurse and physician to estimate when the tubing was inserted into the patient. Due to this estimation, medical risks are experienced with the replacement of the tubing that may be removed or replaced much later than needed.

Despite this wide variety of commercially available label types, there is a current need for medical labels or tags that protect the information, particularly the date information, written on the tag. There is a current need for a label or tag that protects the date information on the label or tag so that a physician or nurse knows when to replace the tubing on which the label or tag is attached. Further, there is a current need for a label or tag that protects the date information from deterioration on the label or tag so that a nurse or physician can easily identify when the tubing must be replaced. Further, there is a current need for a label or tag that protects the date information from bodily fluids, including blood, alcohol, food and beverages, and other materials that are commonly spilled on labels or tags.

### SUMMARY OF THE INVENTION

In view of the foregoing, the present disclosure is directed to a protected date tag for medical tubing that solves the aforementioned deficiencies in current medical tubing adhesive labels.

In one example of the present disclosure, a tag for medical tubing may include a transparent adhesive layer, a paper layer positioned on an upper surface of the transparent adhesive layer, and a non-transparent layer positioned on the upper surface of the transparent adhesive layer and an upper surface of the paper layer, wherein a width of the paper layer is less than a width of the transparent adhesive layer such that a portion of the transparent adhesive layer is foldable to overlap the paper layer.

In another example of the present disclosure, the transparent adhesive layer may include an adhesive on the upper surface thereof. The paper layer may include a label to indicate date information. The tag may be configured for application to an outer surface of the medical tubing. At least one of the transparent adhesive layer, the paper layer, and the non-transparent layer may be made of an anti-microbial material. The transparent adhesive layer, the paper layer, and the non-transparent layer may be made of an anti-microbial material. The tag may be substantially rectangular.

In another example of the present disclosure, a medical tubing assembly may include a medical tubing, and a tag adhesively applied to an outer circumferential surface of the medical tubing, the tag may include a transparent adhesive layer, a paper layer positioned on an upper surface of the transparent adhesive layer, and a non-transparent layer positioned on the upper surface of the transparent adhesive layer and an upper surface of the paper layer, wherein a width of the paper layer is less than a width of the transparent adhesive layer such that a portion of the transparent adhesive layer is foldable to overlap the paper layer.

In another example of the present disclosure, the transparent adhesive layer may include an adhesive on the upper surface thereof. The medical tubing may be positioned on the adhesive of the transparent adhesive layer and the portion of the transparent adhesive layer is folded over the medical tubing so that the portion of the transparent adhesive layer overlaps the paper layer. The paper layer may include a label to indicate date information. The tag may be configured for application to an outer surface of the medical tubing. At least one of the transparent adhesive layer, the paper layer, and the non-transparent layer may be made of an anti-microbial material. The transparent adhesive layer, the paper layer, and the non-transparent layer may be made of an anti-microbial material. The tag may be substantially rectangular.

In another example of the present disclosure, a method of applying a tag to a medical tubing may include providing the medical tubing, providing the tag to adhesively apply to the medical tubing, positioning an outer circumferential surface of the medical tubing on an adhesive portion of the tag, folding the adhesive portion of the tag around the outer circumferential surface of the medical tubing, and overlapping the adhesive portion of the tag over a date label on the tag.

These and other features and characteristics of the protected date tag, as well as the methods of operation and functions of the related elements of the tag, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only, and are not intended as a definition of the limits of the disclosure. As used in the specification and claims, the singular form of “a”, “an”, and “the” include plural referents unless the context clearly dictates otherwise

### BRIEF DESCRIPTION OF THE DRAWINGS

FIG. 1 is a top view of a protected date tag according to one example of the present disclosure;

FIG. 2 is an assembly view of the protected date tag of FIG. 1;

FIG. 3 is a cross-sectional view of the protected date tag of FIG. 1;

FIG. 4 is a top view of the protected date tag of FIG. 1 with a non-transparent layer removed;

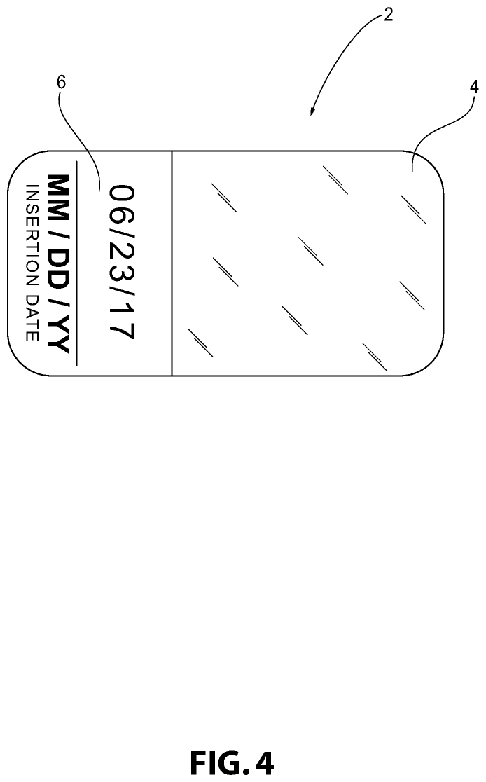

FIG. 5 is a front perspective view of the protected date tag of FIG. 1 before application to medical tubing;

FIG. 6 is a front perspective view of the protected date tag of FIG. 1 after application to the medical tubing; and

FIG. 7 is a top view of a protected date tag according to another example of the present disclosure.

### DESCRIPTION OF THE DISCLOSURE

For purposes of the description hereinafter, the terms “upper”, “lower”, “right”, “left”, “vertical”, “horizontal”, “top”, “bottom”, “lateral”, “longitudinal”, and derivatives thereof, shall relate to the disclosed apparatus as it is oriented in the figures. However, it is to be understood that the apparatus of the present disclosure may assume alternative variations and step sequences, except where expressly specified to the contrary. It is also to be understood that the specific systems and processes illustrated in the attached drawings, and described in the following specification, are simply exemplary examples of the apparatus disclosed herein. Hence, specific dimensions and other physical characteristics related to the examples disclosed herein are not to be considered as limiting.

Referring to the drawings in which like reference numerals refer to like parts throughout the several views thereof, the present disclosure is generally directed to a tag for medical tubing and, more particularly, to a protected date tag for medical tubing. It is to be understood, however, that the protected date tag may be used in many different settings other than medical tubing, including medical containers such as syringe, medical containers, such as tubes, and other medical devices that require information that should be protected from deterioration.

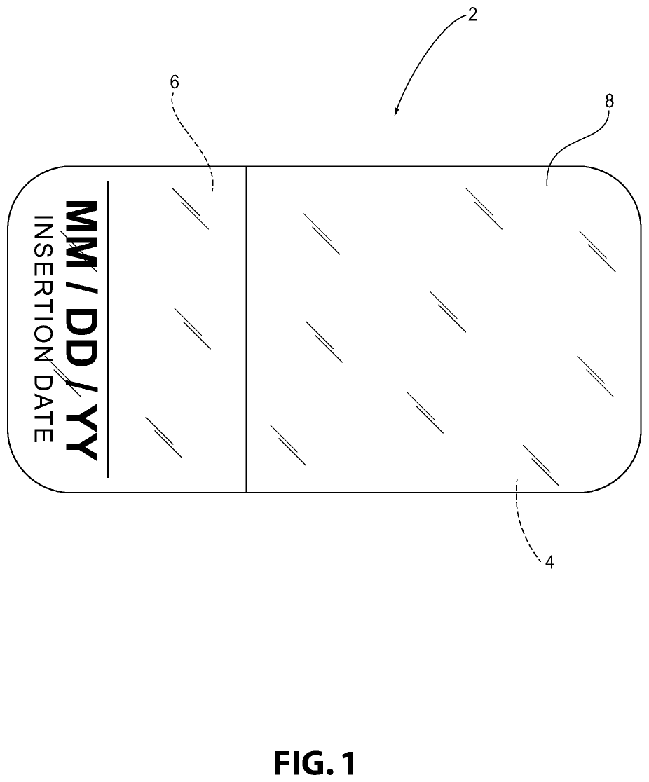

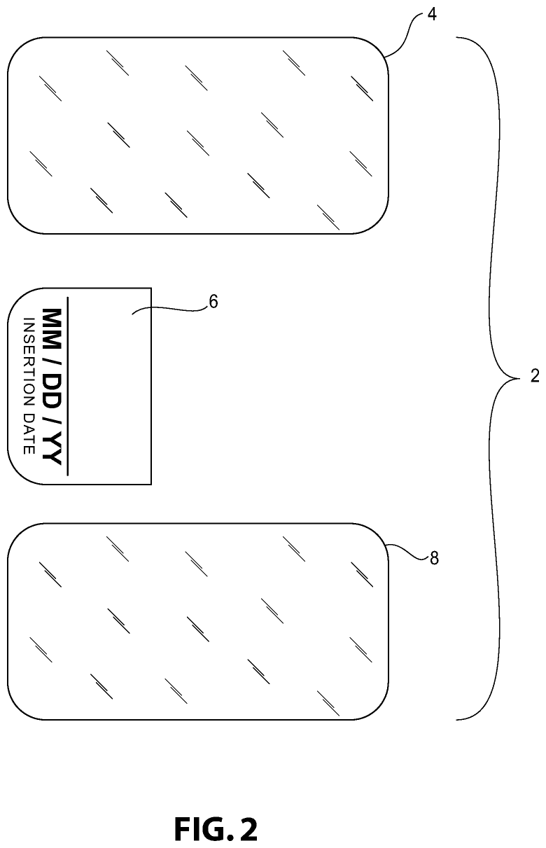

With reference to FIGS. 1 and 2, a tag 2 for medical tubing is shown and described. The tag 2 is generally rectangular in shape. However, it is to be understood that the tag 2 may be of any suitable shape based on the needs of the nurse or physician using the tag 2. In one example, the corners of the tag 2 are rounded. In one example, the tag 2 is a multi-layer tag that is adhesively applied to medical tubing. In some examples, the medical tubing may be catheter tubing, IV tubing, mid-line tubing, nasogastric tubing, nephrostomy tubing, or any other medical tubing on which a tag, such as tag 2, can be applied to supply information regarding the tubing.

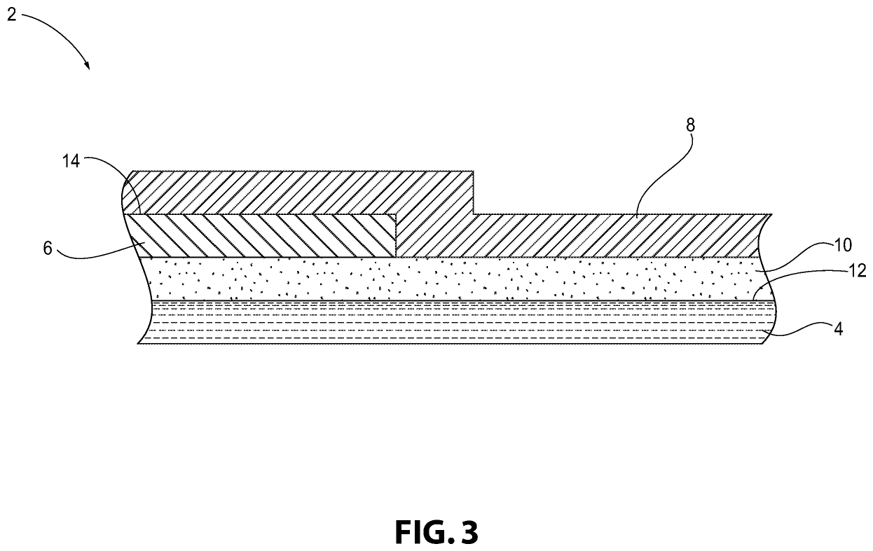

As shown in FIGS. 2 and 3, the tag 2 may include a transparent adhesive layer 4, a paper layer 6, and a non-transparent layer 8. The transparent adhesive layer 4 may be clear and flexible to permit the tag 2 to be bent or flexed around a medical tubing. The transparent adhesive layer 4 may include an adhesive 10 on a top surface 12 thereof. The transparent adhesive layer 4 extends the entire width of the tag 2. In one example, the paper layer 6 is attached to the adhesive 10 of the transparent adhesive layer 4. The paper layer 6 may be white and non-transparent. The paper layer 6 may be made of a paper material such that a nurse or physician can write on the top surface 14 of the paper layer 6. In one example, the paper layer 6 does not extend the entire width of the tag 2. In one example, the paper layer 6 extends less than 50% across the width of the tag 2. In one example, the width of the transparent adhesive layer 4 may be 1.5 inches, while the width of the paper layer 6 may be 0.5 inches. As shown in FIG. 2, in one example of the present disclosure, a “MM/DD/YY” label may be printed on the paper layer 6 to allow a nurse or physician to write thereon a date that the tag 2 was applied to the medical tubing or a date on which the medical tubing should be removed from the patient. In one example of the present disclosure, the non-transparent layer 8 may be applied to the top surface 14 of the paper layer 6 and the top surface 12 of the transparent adhesive layer 4. The non-transparent layer 8 may be made of paper or a flexible plastic. The non-transparent layer 8 is configured to cover and protect the transparent adhesive layer 4 and the paper layer 6 until the nurse or physician is ready to apply the tag 2 to the medical tubing. In one example, the non-transparent layer 8 is configured to removably stick to the adhesive 10 on the transparent adhesive layer 4. Therefore, the non-transparent layer 8 is not permanently applied to the transparent adhesive layer 4. Due to the use of the tag 2 on medical tubing, medical devices, and/or medical containers, in one example, the layers 4, 6, 8 of the tag 2 are made of a medical-grade material that is impermeable to blood and alcohol. In one example, the layers 4, 6, 8 of the tag 2 are made of an anti-microbial material. The layers 4, 6, 8 may be flexible, may be sized to allow the tag 2 to be positioned in a location that makes it easy for a nurse or practitioner to see, but does not bother the patient, may be latex-free so that the tag 2 does not cause an allergic reaction with the patient, may be soft so the tag 2 does not irritate the patient's skin, and may be adhesively secured in place so the tag 2 does not move while the tag 2 is applied to the medical instrument or container.

With reference to FIG. 2, the assembly of the tag 2 is shown and described. After the layers 4, 6, 8 have been manufactured, the paper layer 6 is applied to the adhesive 10 of the transparent adhesive layer 4. In one example, the paper layer 6 is positioned on the transparent adhesive layer 4 so that the outer edge of the paper layer 6 is placed on the outer edge of the transparent adhesive layer 4. After the paper layer 6 has been applied to the transparent adhesive layer 4, the non-transparent layer 8 is positioned over the non-transparent layer 4 and the paper layer 6. FIG. 1 depicts the assembled tag 2.

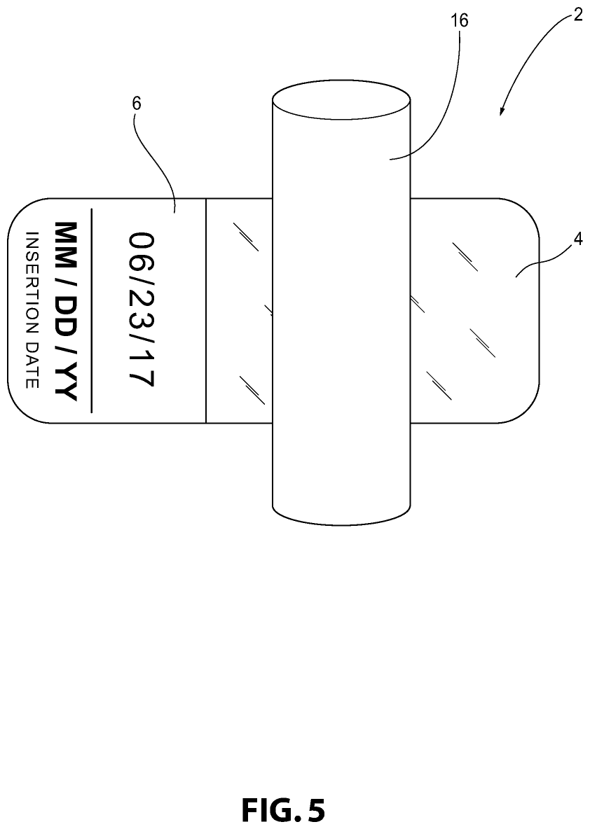

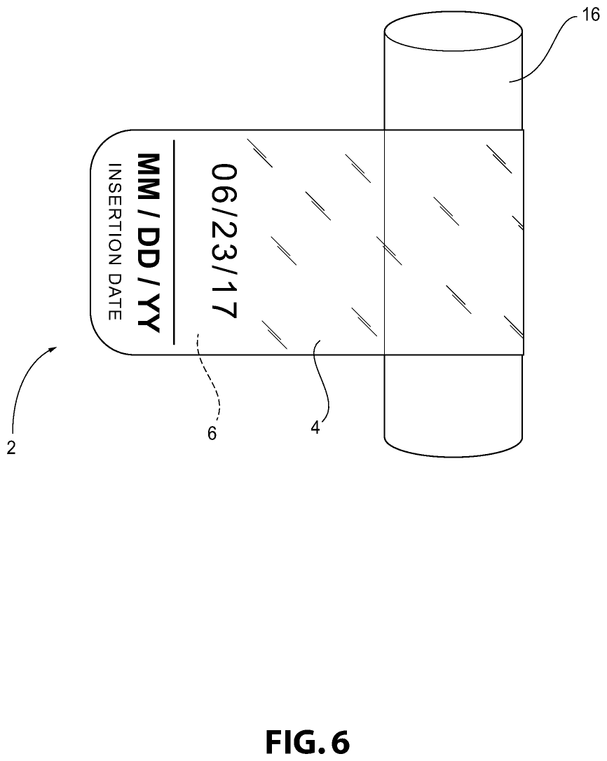

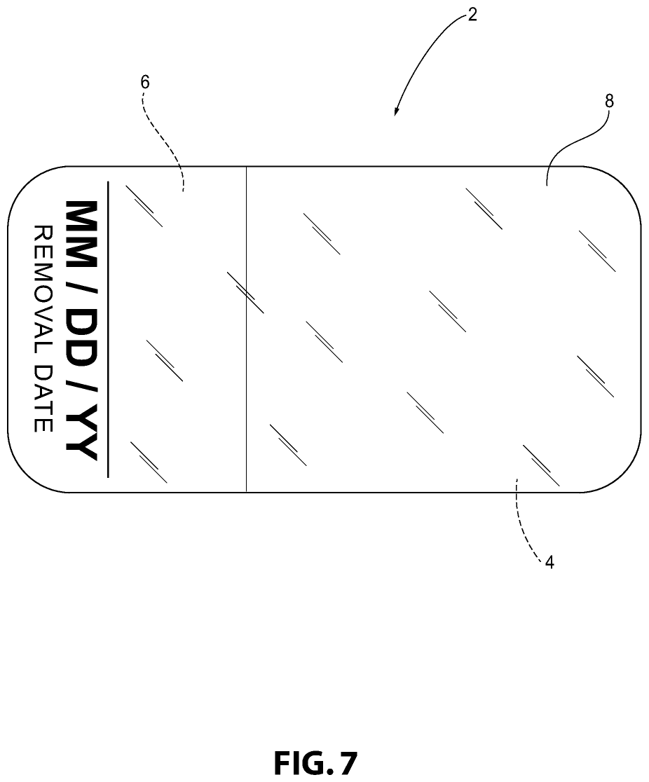

With reference to FIGS. 5 and 6, application of the tag 2 to medical tubing 16 is shown and described. Initially, the nurse or physician will remove the non-transparent layer 8 from the assembled tag 2. The nurse or physician will peel off the non-transparent layer 8 to reveal the paper layer 6 and the transparent adhesive layer 4. After the non-transparent layer 8 has been removed, the nurse or physician will write a date on the label of the paper layer 6. As discussed, the date may be the date that the tag 2 was applied to the medical tubing 16 (see FIG. 1 in which the tag 2 includes an “Insertion Date”) or a date on which the medical tubing 16 should be removed from the patient (see FIG. 7 in which the tag includes a “Removal Date”). After the date has been written on the paper layer 6, the upper surface 12 of the non-transparent layer 4 is placed against the medical tubing 16. Due to the adhesive 10 on the upper surface 12, the tag 2 is adhesively attached to an outer surface of the medical tubing 16. As shown in FIG. 5, the medical tubing 16 is positioned adjacent the paper layer 6 on the tag 2 so that the paper layer 6 is positioned on the left side of the medical tubing 16 and the transparent adhesive layer 4 is positioned on the right side of the medical tubing 16. After the transparent adhesive layer 4 has been applied to the medical tubing 16, the right side end of the transparent adhesive layer 4 is folded around the outer surface of the medical tubing 16 so that the transparent adhesive layer 4 covers the outer circumferential surface of the medical tubing 16. The end of the transparent adhesive layer 4 is placed over and applied to the upper surface 14 of the paper layer 6. The adhesive 10 of the transparent adhesive layer 4 is applied to the upper surface 14 of the paper layer 6 so that the two layers 4, 6 are attached to one another. Due to the transparency of the transparent adhesive layer 4, the date written on the label of the paper layer 6 can be viewed through the transparent adhesive layer 4. The transparent adhesive layer 4 is also configured to protect the written information on the paper layer 6 so that the date written on the paper layer 6 is not smeared, erased, wiped, or deteriorated from the paper layer 6 leaving the nurse or physician to estimate the date that was written on the paper layer 6. By protecting the date information written on the paper layer 6, the nurse or physician can easily identify when the medical tubing 16 was inserted into the patient or when the medical tubing 16 should be replaced in the patient.

While several examples of a protected date tag were shown in the accompanying figures and described in detail hereinabove, other examples will be apparent to, and readily made by, those skilled in the art without departing from the scope and spirit of the disclosure. Accordingly, the foregoing description is intended to be illustrative rather than restrictive. The apparatus of the present disclosure described hereinabove is defined by the appended claims and all changes to the disclosed apparatus that fall within the meaning and range of equivalency of the claims are to be embraced within their scope.

The invention claimed is:

1. A tag for medical tubing, comprising:
a transparent adhesive layer;
a paper layer positioned on an upper surface of the transparent adhesive layer; and
a non-transparent layer positioned on the upper surface of the transparent adhesive layer and an upper surface of the paper layer,
wherein a width of the paper layer is less than a width of the transparent adhesive layer such that a portion of the transparent adhesive layer is foldable to overlap the paper layer.

2. The tag as claimed in claim 1, wherein the transparent adhesive layer comprises an adhesive on the upper surface thereof.

3. The tag as claimed in claim 1, wherein the paper layer comprises a label to indicate date information.

4. The tag as claimed in claim 1, wherein the tag is configured for application to an outer surface of the medical tubing.

5. The tag as claimed in claim 1, wherein at least one of the transparent adhesive layer, the paper layer, and the non-transparent layer is made of an anti-microbial material.

6. The tag as claimed in claim 1, wherein the transparent adhesive layer, the paper layer, and the non-transparent layer are made of an anti-microbial material.

7. The tag as claimed in claim 1, wherein the tag is substantially rectangular.

8. A medical tubing assembly, comprising:
a medical tubing; and
a tag adhesively applied to an outer circumferential surface of the medical tubing, the tag comprising:
a transparent adhesive layer;
a paper layer positioned on an upper surface of the transparent adhesive layer; and
a non-transparent layer positioned on the upper surface of the transparent adhesive layer and an upper surface of the paper layer,
wherein a width of the paper layer is less than a width of the transparent adhesive layer such that a portion of the transparent adhesive layer is foldable to overlap the paper layer.

9. The medical tubing assembly as claimed in claim 8, wherein the transparent adhesive layer comprises an adhesive on the upper surface thereof.

10. The medical tubing assembly as claimed in claim 9, wherein the medical tubing is positioned on the adhesive of the transparent adhesive layer and the portion of the transparent adhesive layer is folded over the medical tubing so that the portion of the transparent adhesive layer overlaps the paper layer.

11. The medical tubing assembly as claimed in claim 8, wherein the paper layer comprises a label to indicate date information.

12. The medical tubing assembly as claimed in claim 8, wherein the tag is configured for application to an outer surface of the medical tubing.

13. The medical tubing assembly as claimed in claim 8, wherein at least one of the transparent adhesive layer, the paper layer, and the non-transparent layer is made of an anti-microbial material.

14. The medical tubing assembly as claimed in claim 8, wherein the transparent adhesive layer, the paper layer, and the non-transparent layer are made of an anti-microbial material.

15. The medical tubing assembly as claimed in claim 8, wherein the tag is substantially rectangular.

16. A method of applying a tag to a medical tubing, comprising:
providing the medical tubing;
providing the tag to adhesively apply to the medical tubing;
positioning an outer circumferential surface of the medical tubing on an adhesive portion of the tag;
folding the adhesive portion of the tag around the outer circumferential surface of the medical tubing; and
overlapping the adhesive portion of the tag over a date label on the tag.
